(19) United States
(12) Patent Application Publication
(10) Pub. No.: US 2023/0047251 A1
(43) Pub. Date: Feb. 16, 2023

(54) ALLOSTERIC MODULATORS OF CANNABINOID RECEPTORS AND METHODS OF TREATING NEUROPATHIC PAIN

(71) Applicant: The Board of Trustees of the Leland Stanford Junior University, Stanford, CA (US)

(72) Inventors: Sanjay Malhotra, Palo Alto, CA (US); Brian K. Kobilka, Palo Alto, CA (US); Kaavya Krishna Kumar, Stanford, CA (US); Angel Resendez, Palo Alto, CA (US)

(21) Appl. No.: 17/792,665

(22) PCT Filed: Feb. 3, 2021
(86) PCT No.: PCT/US2021/016431

§ 371 Date: Jul. 13, 2022

Related U.S. Application Data
(60) Provisional application No. 62/975,633, filed on Feb. 12, 2020.

(51) Int. Cl. A61K 31/4439; A61K 45/06; A61P 25/02; A61P 25/04; A61K 31/404

(52) U.S. Cl. CPC A61K 31/4439; A61K 31/404; A61K 45/06; A61P 25/02; A61P 25/04

(57) ABSTRACT

Provided are methods for treating neuropathic pain by administering to a subject in need thereof a compound comprising phenyl-indole scaffold, wherein the compound has a modulatory effect on cannabinoid type 1 receptor (CB₁R). Pharmaceutical compositions containing compounds comprising phenyl-indole scaffolds and pharmaceutically acceptable carriers are also provided, along with methods of using the same.

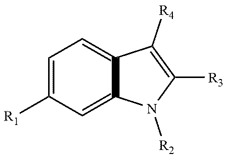

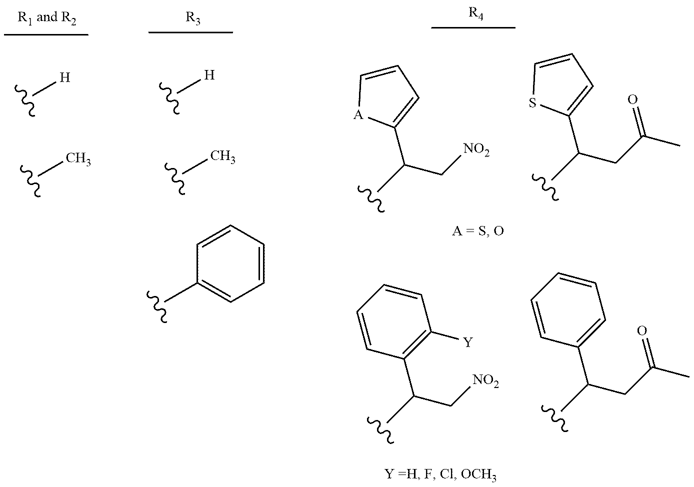

### CROSS-REFERENCING

This application claims the benefit of U.S. Provisional Application Serial No. 62/975,633, filed on Feb. 12, 2020, which application is incorporated by reference herein.

### INTRODUCTION

Neuropathic pain is a complex, chronic pain state arising from a lesion or disease affecting the somatosensory nervous system and is common to conditions such as back and neck pain, diabetic peripheral neuropathy, and fibromyalgia. Neuropathic pain is estimated to affect 1 in 10 adults over the age of 30, is associated with a 3-fold increase in direct healthcare costs and contributes significantly to the $100 billion annual indirect costs attributed to chronic pain conditions due to absenteeism and decreased productivity.

The current first-line treatments for neuropathic pain are tricyclic antidepressants (nortriptyline, desipramine) and anticonvulsants (gabapentin, pregabalin); however, only 30% of sufferers experience even partial relief of symptoms from these drugs, and 30% report dose-limiting side effects. Opioid drugs are a second-line treatment for neuropathic pain, since they are less effective for neuropathic pain than for nociceptive pain, and chronic use is associated with adverse reactions, tolerance, and addiction.

The prevalence, cost, and lack of efficacious treatments make neuropathic pain one of the largest unmet clinical needs today, and new targets for drug development are desperately sought. Functionally selective stimulation of the cannabinoid type 1 receptor (CB1R), one of the most abundantly expressed G protein-coupled receptors (GPCRs) in the central nervous systems (CNS), has been proposed as a promising platform for the treatment of neuropathic pain refractory to current therapeutics.

Many CB1R agonists demonstrate efficacy in preclinical rodent models or clinical neuropathic pain sufferers, including endogenous ligand anandamide (AEA), exogenous agonist Δ9-tetrahydrocannabinol (THC, found in cannabis), and many synthetic cannabinoids. However, activation of CB1R via its orthosteric site is associated with side-effects including intoxication and hypothermia. Despite demonstrating efficacy in numerous clinical trials for neuropathic pain, broad therapeutic application of cannabinoid agonists is hindered by dose limiting psychotropic effects and tolerance.

Agonist-mediated activation of CB1R at the orthosteric site leads to a diverse array of signaling mechanisms predominantly via G proteins of the Gi/o family (Gi1, 2, and 3, and Go1, and 2), phosphorylation of extracellular signal-regulated kinases (ERK), and β-arrestin recruitment. Although selective activation of CB1R signaling pathways could mitigate adverse effects, most orthosteric agonists (like AEA and THC) induce active CB1R conformations that demonstrate little signaling bias. The rational design of functionally selective orthosteric CB1R agonists with reduced side effects for the treatment of neuropathic pain is difficult in the absence of crystallographic CB1R structural information.

A small molecule (ZCZ-011) targeting a purported allosteric site on CB1R could treat a rodent model of neuropathic pain without psychoactive or hypothermic effects. This suggests that positive allosteric modulators (PAMs) of CB1R may induce active conformations of the receptor that increase the tone of basal endocannabinoid CB1R signaling in a functionally selective way that minimizes adverse effects.

Few CB1R PAMs are known, and these typically enhance binding of orthosteric agonists while simultaneously decreasing signaling efficacy, behaving as functionally insurmountable antagonists. Additionally, CB1R negative allosteric modulators display contradictory effects in different assays, further confounding the rational development of ligands targeting the CB1R allosteric site. Innovative methods are required for screening libraries of potential functionally selective agonists and PAMs of CB1R for their ability to enhance selective signaling efficacy. In particular, since the β-arrestin signaling pathway is associated with desensitization, CB1R internalization, and tolerance, new methods for identifying biased PAMs capable of enhancing G protein signaling rather than β-arrestin recruitment would expedite the rational design of new therapeutic candidates for the treatment of neuropathic pain.

### SUMMARY

Certain embodiments of this disclosure provide compounds that are biased PAMs capable of enhancing G protein signaling rather than β-arrestin and, thus, could be used for the treatment of neuropathic pain. Certain such compounds include the compound of Formula I:

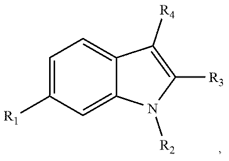

wherein:
R₁ is H, C₁-C₆ alkyl, aliphatic, alkoxy, amide, amine, thioether, haloalkyl, nitro, halogen, cycloaliphatic, or aryl;
R₂ is H, C₁-C₆ alkyl, aliphatic, alkoxy, amide, amine, thioether, haloalkyl, nitro, halogen, cycloaliphatic, or aryl;
R₃ is H, C₁-C₆ alkyl, or substituted or unsubstituted monocyclic or bicyclic aromatic ring containing 5 to 10 carbon atoms, wherein, one or more carbon atoms are replaced with one or more of N, O, or S; and
R₄ is C₁-C₆ nitro-alkyl, C₁-C₆ alkyl-amine, C₁-C₆ ketone, C₁-C₆ aldehyde, or C₁-C₆ carbonyl, wherein one or more carbons of the alkyl chain are substituted with substituted or unsubstituted monocyclic or bicyclic aromatic ring containing 5 to 10 carbon atoms, wherein, one or more carbon atoms are replaced with one or more of N, O, or S, and
wherein, the monocyclic or bicyclic aromatic ring is further optionally substituted with one or more groups selected from halogen, C₁-C₆ alkyl, alkoxy, carbonyl, amine, alkyl-amine, nitro, and nitro-alkyl.

In preferred embodiments, such compounds include the compounds described in FIG. 1.

These compounds can be used to treat neuropathic pain and, accordingly, certain embodiments of this disclosure provide a method of treating neuropathic pain by administering a therapeutically effective amount of the compounds disclosed herein.

Further embodiments of this disclosure also provide pharmaceutical compositions comprising the compounds disclosed herein and a pharmaceutically acceptable carrier.

### BRIEF DESCRIPTION OF THE DRAWINGS

FIG. 1. PAM chemical library.

FIG. 2. Luciferase assay for evaluating GTP-turnover.

FIG. 3. GTP turnover assay Characterization results of new PAMS as potential G-protein activators (couple) independently or in the presence of an orthosteric ligand CP 55,940.

FIG. 4. Synthetic scheme for the generation of enantiopure 2-substituted indoles.

FIG. 5. β-arrestin recruitment assay.

FIG. 6A. FLARE assay showing β-arrestin-2 coupling, increase in Luciferase is associated with an increase in arrestin 2 coupling.

FIG. 6B. GTP-turnover assay showing Gi coupling, decrease in Luciferase is associated with an increase in Gi coupling.

### DETAILED DESCRIPTION

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” “comprise,” “consisting essentially of,” “consists essentially of,” "consisting," and "consists" can be used interchangeably.

The phrases “consisting essentially of” or “consists essentially of” indicate that the claim encompasses embodiments containing the specified materials or steps and those that do not materially affect the basic and novel characteristic(s) of the claim.

“Treatment” or “treating” (and grammatical variants of these terms), as used herein refer to an approach for obtaining beneficial or desired results including but not limited to therapeutic benefit, such as decrease in neuropathic pain. A therapeutic benefit is achieved with the eradication or amelioration of one or more of the physiological symptoms associated with the underlying disorder such that an improvement is observed in the patient, notwithstanding that the patient may still be afflicted with the underlying disorder. A therapeutic effect includes delaying the appearance of neuropathic pain, delaying the onset of symptoms of neuropathic pain, slowing, halting, or reversing the progression of neuropathic pain or any combination thereof.

The term “therapeutically effective amount” refers to that amount of a compound described herein that is sufficient to affect the intended application including but not limited to disease treatment. The therapeutically effective amount may vary depending upon the intended application, the subject, e.g., the weight and age of the subject, and disease condition being treated, e.g., the severity of the disease condition, the manner of administration and the like. The specific dose will vary depending on the particular compounds chosen, the dosing regimen to be followed, whether it is administered in combination with other compounds, timing of administration, the tissue to which it is administered, and the physical delivery system in which it is carried.

A “sub-therapeutic amount” of an agent is an amount less than the effective amount for that agent, but which when combined with an effective or sub-therapeutic amount of another agent or therapy can produce a desired result, due to, for example, synergy in the resulting efficacious effects (e.g., therapeutic benefit) for the subject, or reduced side effects associated with the compounds administered to the subject. Typical therapeutic amounts for an agent, as disclosed herein, can be ascertained from various publicly available sources (e.g., drugs.com, The Physician’s Desk Reference, or scientific literature). Subtherapeutic amounts of an agent, as provided herein, are amounts less than those reported in the publicly available sources as the therapeutically effective amounts. Subtherapeutic amount of an agent is preferably, less than 80%; more preferably, less than 70%; even more preferably, less than 60%; and most preferably, less than 50% of the therapeutic amount.

A “synergistically effective” amount of a compound disclosed herein is an amount which, when combined with an effective or subtherapeutic amount of another agent or therapy, produces a greater effect than when either of the two agents are used alone. A synergistically effective therapeutic amount of an agent produces a greater effect when used in combination than the additive effects of each of the two agents or therapies when used alone. The term “greater effect” encompasses not only a reduction in symptoms of the disorder to be treated, but also an improved side effect profile, improved tolerability, improved patient compliance, improved efficacy, or any other improved clinical outcome.

The terms “co-administration,” “administered in combination with,” and their grammatical equivalents encompass administration of two or more agents to a subject so that both therapeutic agents and/or their metabolites are present in the subject at the same time. Co-administration includes simultaneous administration in separate compositions, administration at different times in separate compositions, or administration in a composition in which both agents are present. Co-administered agents may be in the same formulation.

The term “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the compositions.

“Subject” refers to an animal, such as a mammal, for example a human. The methods described herein can be useful in both pre-clinical human therapeutics and veterinary applications.

The terms “simultaneous” or “simultaneously” as applied to administering agents to a subject refer to administering one or more agents at the same time, or at two different time points that are separated by no more than 1 hour. The term “sequentially” refers to administering more than one agent at two different time points that are separated by more than 1 hour, e.g., about 2 hours, about 5 hours, 8 hours, 1 day, 2 days, 3 days, 4 days, 5 days, 6 days, 7 days or even longer.

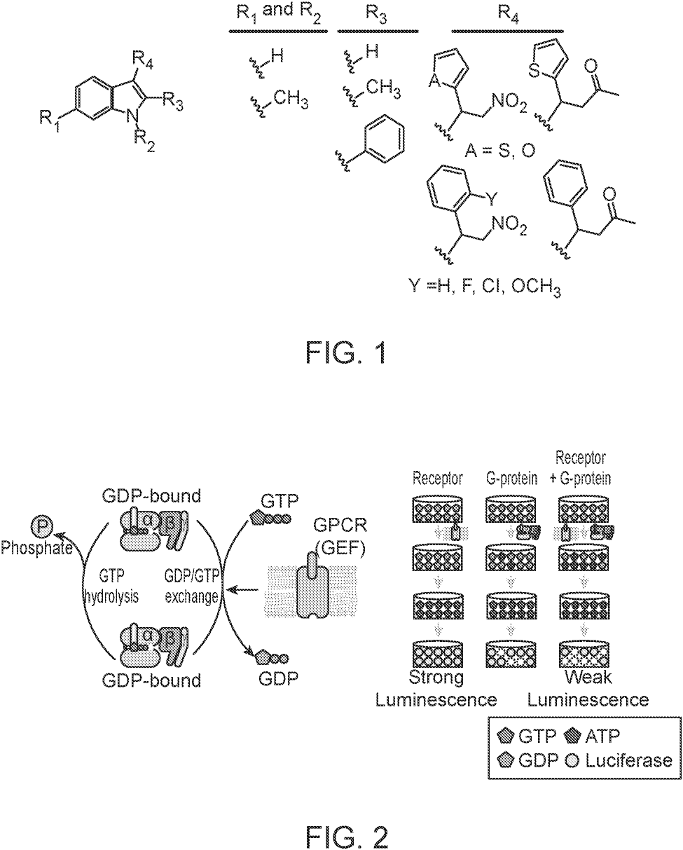

A chemical library of novel compounds was designed/synthesized around phenyl-indole scaffold. The compounds were characterized for their modulatory effect on CB1R, and structure-activity relationships (SARs) was systematically explored within this class of CB1R PAMs. A synthetic method was developed where mono-substituted indoles were subjected to direct, palladium-catalyzed C-2 arylation, followed by Friedel-Crafts type alkylation with judiciously substituted nitroalkene. Introduction of the nitro group served as a convenient synthon for further structural elaboration. Structurally diverse members of this chemotype were designed and synthesized, for example, as depicted in FIG. 1.

The newly synthesized compounds were tested for their ability to activate G-protein activation rather than β-arrestin recruitment. Such activity was observed and, thus, these compounds could be used for the treatment of neuropathic pain.

Accordingly, certain embodiments of the invention provide a method for treating neuropathic pain in a subject by administering to the subject a therapeutically effective amount of a compound of Formula I:

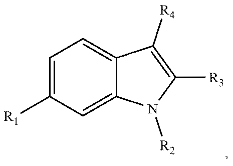

wherein:
R₁ is H, C₁-C₆ alkyl, aliphatic, alkoxy, amide, amine, thioether, haloalkyl, nitro, halogen, cycloaliphatic, or aryl;
R₂ is H, C₁-C₆ alkyl, aliphatic, alkoxy, amide, amine, thioether, haloalkyl, nitro, halogen, cycloaliphatic, or aryl;
R₃ is H, C₁-C₆ alkyl, or substituted or unsubstituted monocyclic or bicyclic aromatic ring containing 5 to 10 carbon atoms, wherein, one or more carbon atoms are replaced with one or more of N, O, or S; and
R₄ is C₁-C₆ nitro-alkyl, C₁-C₆ alkyl-amine, C₁-C₆ ketone, C₁-C₆ aldehyde, or C₁-C₆ carbonyl, wherein one or more carbons of the alkyl chain is substituted with substituted or unsubstituted monocyclic or bicyclic aromatic ring containing 5 to 10 carbon atoms, wherein, one or more carbon atoms are replaced with one or more of N, O, or S, and
wherein, the monocyclic or bicyclic aromatic ring is further optionally substituted with one or more groups selected from halogen, C₁-C₆ alkyl, alkoxy, carbonyl, amine, alkyl-amine, nitro, and nitro-alkyl.

In preferred embodiments, in the compound of Formula I:
R₁ is H or C₁-C₆ alkyl;
R₂ is H or C₁-C₆ alkyl;
R₃ is H, C₁-C₆ alkyl, pyridinyl, or phenyl; and
R₄ is nitro-alkyl, ketone, or aldehyde.

In the compounds of Formula I, the monocyclic or bicyclic aromatic ring containing 5 to 10 carbon atoms in the R₃ or R₄ position can be phenyl, naphthyl, pyridine, quinoline, isoquinoline, pyrazine, quinoxaline, pyrimidine, quinazoline, pyridazine, cinnoline, phthalazine, triazine, furan, benzofuran, isobenzofuran, pyrrole, indole, isoindole, thiophene, benzothiophene, imidazole, benzimidazole, purine, pyrazole, indazole, oxazole, benzoxazole, isoxazole, benzisoxazole, thiazole, or benzothiazole.

In particularly preferred methods, in the compound of Formula I:
R₁ is H or methyl;
R₂ is H or methyl;
R₃ is H, methyl, pyridinyl, or phenyl; and
R₄ is:
nitro-ethyl substituted with phenyl, furan, or thiophene, optionally substituted with halogen or alkoxy; or
2-butanone, substituted with phenyl, furan, or thiophene, optionally substituted with halogen or alkoxy.

In the most preferred embodiments, the compound is as shown in FIG. 1. “Neuropathic pain” as used herein refers to chronic pain caused by injury or damage to the nerves in the central nervous system. Neuropathic pain may be associated with abnormal sensations called dysesthesia and pain produced by normally non-painful stimuli (allodynia). Neuropathic pain may have continuous and/or episodic (paroxysmal) components. Common qualities of neuropathic pain include burning or coldness, “pins and needles” sensations, numbness and itching.

Neuropathic pain can be caused by an underlying disorder, including autoimmune diseases such as Sjogren’s syndrome, lupus, rheumatoid arthritis, Guillain-Barre syndrome, chronic inflammatory demyelinating polyneuropathy and vasculitis; diabetes; infections, such as viral or bacterial infections, including Lyme disease, shingles, Epstein-Barr virus, hepatitis B and C, leprosy, diphtheria, and HIV; inherited disorders, such as Charcot-Marie-Tooth disease; tumors; bone marrow disorders, such as an abnormal protein in the blood (monoclonal gammopathies), a form of bone cancer (myeloma), lymphoma and the rare disease amyloidosis; and other diseases including kidney disease, liver disease, connective tissue disorders and an underactive thyroid (hypothyroidism). Other causes of neuropathies also include alcoholism; poor dietary choices made by people with alcoholism can lead to vitamin deficiencies; exposure to poisons; certain medications, such as those used to treat cancer (chemotherapy); trauma or pressure on the nerve, such as traumas from motor vehicle accidents, falls or sports injuries that sever or damage peripheral nerves; nerve pressure from having a cast or using crutches or repeating a motion such as typing many times; or vitamin deficiencies, such as deficiency of B-1, B-6 and B-12 — vitamin E and niacin. In a number of cases, no cause can be identified and neuropathic pain is idiopathic.

The compounds disclosed herein can be administered locally or systemically. Systemic administration can be achieved by administration routes including parenteral routes such as intravenous, intraarterial, subcutaneous, transdermal, intradermal, intramuscular, intraperitoneal, nasal, intracranial, intrathecal, intracardiac, intraosseous or transmucosal routes or enteral routes, such as oral, rectal, sublingual, or buccal. Local administration can be achieved by administration routes including topical, epidural, epicutaneous, inhalational, nasal, intraarticular, vaginal, auricular, intravitreal routes, or intracranial.

For a prolonged administration of the compounds disclosed herein at a predetermined rate the pharmaceutical compositions can also be administered in sustained or controlled release dosage forms, such as depot injections or osmotic pumps.

In certain embodiments, the compounds disclosed herein after administered in combination with another agent or therapy that is also suitable for the treatment of neuropathic pain. In certain such embodiments, the compound disclosed herein and/or the other agent or therapy is administered in a subtherapeutic amount. This can lead to therapeutic effect while minimizing or avoiding adverse side effects of the compound or the other therapy.

Such other therapies include analgesics, anticonvulsants, tricyclic antidepressants (TCAs), selective serotonin-norepinephrine reuptake inhibitors, topical anesthetic agents, nonsteroidal anti-inflammatory drugs (NSAIDs), antiarrhythmics, narcotic analgesics, and opioids.

Non-limiting examples of TCAs include amitriptyline, desipramine, doxepin, imipramine, nortriptyline, and trimipramine. Non-limiting examples of selective serotonin-norepinephrine reuptake inhibitors include venlafaxine, desvenlafaxine, duloxetine, milnacipran, and levomilnacipran. Non-limiting examples of anticonvulsants include gabapentin, pregabalin, carbamazepine, oxcarbazepine, topiramate, and lamotrigine. Non-limiting examples of opioids include tramadol, tapentadol, methadone, and levorphanol. Additional examples of other therapies against neuropathic pain are known in the art and such embodiments are within the embodiments of the invention.

A pharmaceutically effective dose of the compounds disclosed herein can be between: 0.01 and 20 mg/kg per day, 0.1 and 15 mg/kg per day, 1 to 10 mg/kg per day, 5 to 10 mg/kg per day, 6 to 9 mg/kg per day, or 7 to 8 mg per day. When the compounds disclosed herein are used in combination with another agent for the treatment of neuropathic pain, the amount of the compound administered is less than the therapeutic amount, preferably, less than 80%; more preferably, less than 70%; even more preferably, less than 50%; and most preferably, less than 50% of the therapeutic amount.

### Pharmaceutical Compositions and Dosages

Further embodiments of the invention provide pharmaceutical compositions of the compounds disclosed herein. Such compositions comprise the compounds disclosed herein and a pharmaceutically acceptable carrier. The compositions can further include an additional agent for the treatment of neuropathic pain, for example, the additional agents disclosed above, such as analgesics, anticonvulsants, tricyclic antidepressants (TCAs), selective serotonin-norepinephrine reuptake inhibitors, topical anesthetic agents, nonsteroidal anti-inflammatory drugs (NSAIDs), antiarrhythmics, narcotic analgesics, and opioids.

The pharmaceutical compositions can contain pharmaceutically acceptable excipients, diluents, adjuvants, stabilizers, emulsifiers, preservatives, colorants, buffers, or flavor imparting agents.

Suitable oral compositions can be in the form of tablets, troches, lozenges, aqueous or oily suspensions, dispersible powders or granules, emulsion, hard or soft capsules, syrups or elixirs. Formulations for oral use may also be presented as hard gelatin capsules wherein the compound and/or an additional agent is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose or saccharin. Sweetening agents and flavoring agents may be added to provide palatable oral preparations. These compositions may be preserved by the addition of an anti-oxidant such as ascorbic acid.

Examples of carriers include inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The compositions in a table form may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

For aqueous suspensions the compounds disclosed herein can be mixed with carriers suitable for maintaining a stable suspension. Examples of such carriers include sodium carboxymethylcellulose, methylcellulose, hydropropylmethylcellulose, sodium alginate, polyvinylpyrrolidone, gum tragacanth and gum acacia.

Oily suspensions may be formulated by suspending the compounds in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Dispersible powders and granules suitable for preparation of an aqueous suspension by the addition of water can provide the compounds in admixture with a dispersing or wetting agent, suspending agent and one or more preservatives. Suitable dispersing or wetting agents and suspending agents are exemplified by those already mentioned above.

Pharmaceutical compositions can also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth naturally-occurring phosphatides, for example soybean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic, parentally acceptable diluent or solvent, for example as a solution in 1,3-butanediol. Among the acceptable vehicles and solvents that may be employed are water, Ringer’s solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Compositions for rectal administration can be prepared by mixing the compounds disclosed herein with a suitable non-irritating carrier which is solid at ordinary temperatures but liquid at the rectal temperature and will therefore melt in the rectum to release the compound. Examples of such materials are cocoa butter and polyethylene glycols.

Compositions for parenteral administrations are administered in a sterile medium. Depending on the vehicle used and the concentration of the drug in the formulation, the parenteral formulation can either be a suspension or a solution containing dissolved drug. Adjuvants such as local anesthetics, preservatives and buffering agents can also be added to parenteral compositions.

Parenteral administration can be by bolus injection or by gradual perfusion over time. Preparations for parenteral administration include sterile aqueous or non-aqueous solutions, suspensions, and emulsions, which may contain auxiliary agents or excipients known in the art and can be prepared according to routine methods. In addition, suspension of the active compounds as appropriate oily injection suspensions may be administered. Suitable lipophilic solvents or vehicles include fatty oils, for example, sesame oil, or synthetic fatty acid esters, for example, sesame oil, or synthetic fatty acid esters, for example, ethyl oleate or triglycerides. Aqueous injection suspensions that may contain substances increasing the viscosity of the suspension include, for example, sodium carboxymethyl cellulose, sorbitol, and/or dextran. Optionally, the suspension may also contain stabilizers. Pharmaceutical compositions include suitable solutions for administration by injection, and contain from about 0.01 to 99.99 percent, preferably from about 20 to 75 percent of active compound together with the carrier.

Notwithstanding the appended claims, the present disclosure is further defined by the following embodiments:

Embodiment 1. A method for treating neuropathic pain in a subject, comprising administering to the subject a therapeutically effective amount of a compound of Formula I:

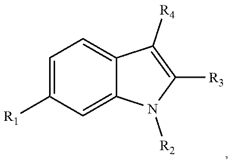

wherein:
R₁ is H, C₁-C₆ alkyl, aliphatic, alkoxy, amide, amine, thioether, haloalkyl, nitro, halogen, cycloaliphatic, or aryl;
R₂ is H, C₁-C₆ alkyl, aliphatic, alkoxy, amide, amine, thioether, haloalkyl, nitro, halogen, cycloaliphatic, or aryl;
R₃ is H, C₁-C₆ alkyl, or substituted or unsubstituted monocyclic or bicyclic aromatic ring containing 5 to 10 carbon atoms, wherein, one or more carbon atoms are replaced with one or more of N, O, or S; and
R₄ is C₁-C₆ nitro-alkyl, C₁-C₆ alkyl-amine, C₁-C₆ ketone, C₁-C₆ aldehyde, or C₁-C₆ carbonyl, wherein one or more carbons of the alkyl chain is substituted with substituted or unsubstituted monocyclic or bicyclic aromatic ring containing 5 to 10 carbon atoms, wherein, one or more carbon atoms are replaced with one or more of N, O, or S; and
wherein, the monocyclic or bicyclic aromatic ring is further optionally substituted with one or more groups selected from halogen, C₁-C₆ alkyl, alkoxy, carbonyl, amine, alkyl-amine, nitro, and nitro-alkyl.

2. The method of claim 1, wherein in the compound of Formula I:
R₁ is H or C₁-C₆ alkyl;
R₂ is H or C₁-C₆ alkyl;
R₃ is H, C₁-C₆ alkyl, pyridinyl, or phenyl; and
R₄ is nitro-alkyl, ketone, or aldehyde.

Embodiment 3. The method of embodiment 1 or 2, wherein in the compound of Formula I the monocyclic or bicyclic aromatic ring containing 5 to 10 carbon atoms in the R₃ or R₄ position is phenyl, naphthyl, pyridine, quinoline, isoquinoline, pyrazine, quinoxaline, pyrimidine, quinazoline, pyridazine, cinnoline, phthalazine, triazine, furan, benzofuran, isobenzofuran, pyrrole, indole, isoindole, thiophene, benzothiophene, imidazole, benzimidazole, purine, pyrazole, indazole, oxazole, benzoxazole, isoxazole, benzisoxazole, thiazole, or benzothiazole.

Embodiment 4. The method of any one of the preceding embodiments, wherein in the compound of Formula I:
R₁ is H or methyl;
R₂ is H or methyl;
R₃ is H, methyl, pyridinyl, or phenyl; and
R₄ is:
nitro-ethyl substituted with phenyl, furan, or thiophene, optionally substituted with halogen or alkoxy; or
2-butanone, substituted with phenyl, furan, or thiophene, optionally substituted with halogen or alkoxy.

Embodiment 5. The method of any one of the preceding embodiments, wherein the compound is as shown in FIG. 1.

Embodiment 6. The method of any one of the preceding embodiments, wherein the neuropathic pain is caused by an autoimmune disease, diabetes, infection, inherited disorder, tumor, bone marrow disorder, kidney disease, liver disease, connective tissue disorder, underactive thyroid (hypothyroidism), trauma or pressure on the nerve, or vitamin deficiency.

Embodiment 7. The method of any one of the preceding embodiments, comprising administering the compound via a route selected from intravenous, intraarterial, subcutaneous, transdermal, intradermal, intramuscular, intraperitoneal, nasal, intracranial, intrathecal, intracardiac, intraosseous, transmucosal, oral, rectal, sublingual, topical, epidural, epicutaneous, inhalational, intraarticular, vaginal, auricular, and intravitreal.

Embodiment 8. The method of any one of the preceding embodiments, further comprising administering to the subject a second agent for the treatment of neuropathic pain.

Embodiment 9. The method of embodiment 8, comprising administering the second agent in a subtherapeutic amount and/or administering the compound of Formula I in a subtherapeutic amount.

Embodiment 10. The method of embodiment 8 or 9, wherein the second agent is an analgesic, anticonvulsant, tricyclic antidepressant, selective serotonin-norepinephrine reuptake inhibitor, topical anesthetic agent, nonsteroidal anti-inflammatory drug, antiarrhythmics, narcotic analgesic, or opioid.

Embodiment 11. A pharmaceutical composition comprising a compound of Formula I and a pharmaceutically acceptable carrier, wherein the compound of Formula I comprises:

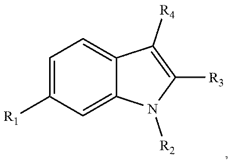

wherein:
R₁ is H, C₁-C₆ alkyl, aliphatic, alkoxy, amide, amine, thioether, haloalkyl, nitro, halogen, cycloaliphatic, or aryl;
R₂ is H, C₁-C₆ alkyl, aliphatic, alkoxy, amide, amine, thioether, haloalkyl, nitro, halogen, cycloaliphatic, or aryl;
R₃ is H, C₁-C₆ alkyl, or substituted or unsubstituted monocyclic or bicyclic aromatic ring containing 5 to 10 carbon atoms, wherein, one or more carbon atoms are replaced with one or more of N, O, or S; and
R₄ is C₁-C₆ nitro-alkyl, C₁-C₆ alkyl-amine, C₁-C₆ ketone, C₁-C₆ aldehyde, or C₁-C₆ carbonyl, wherein one or more carbons of the alkyl chain is substituted with monocyclic or bicyclic aromatic ring containing 5 to 10 carbon atoms, wherein, one or more carbon atoms are replaced with one or more of N, O, or S, and
wherein, the monocyclic or bicyclic aromatic ring is further optionally substituted with one or more groups selected from halogen, C₁-C₆ alkyl, alkoxy, carbonyl, amine, alkyl-amine, nitro, and nitro-alkyl.

Embodiment 12. The pharmaceutical composition of embodiment 11, wherein in the compound of Formula I:
R₁ is H or C₁-C₆ alkyl;
R₂ is H or C₁-C₆ alkyl;
R₃ is H, C₁-C₆ alkyl, pyridinyl, or phenyl; and
R₄ is nitro-alkyl, ketone, or aldehyde.

Embodiment 13. The pharmaceutical composition of embodiment 11 or 12, wherein in the compound of Formula I the monocyclic or bicyclic aromatic ring containing 5 to 10 carbon atoms in the R₃ or R₄ position is phenyl, naphthyl, pyridine, quinoline, isoquinoline, pyrazine, quinoxaline, pyrimidine, quinazoline, pyridazine, cinnoline, phthalazine, triazine, furan, benzofuran, isobenzofuran, pyrrole, indole, isoindole, thiophene, benzothiophene, imidazole, benzimidazole, purine, pyrazole, indazole, oxazole, benzoxazole, isoxazole, benzisoxazole, thiazole, or benzothiazole.

Embodiment 14. The pharmaceutical composition of any one of embodiments 11 to 13, wherein in the compound of Formula I:
R₁ is H or methyl;
R₂ is H or methyl;
R₃ is H, methyl, pyridinyl, or phenyl; and
R₄ is:
nitro-ethyl substituted with phenyl, furan, or thiophene, optionally substituted with halogen or alkoxy; or
2-butanone, substituted with phenyl, furan, or thiophene, optionally substituted with halogen or alkoxy.

Embodiment 15. The pharmaceutical composition of any one of embodiments 11 to 14, wherein the compound is as shown in FIG. 1.

Embodiment 16. The pharmaceutical composition of any one of embodiments 11 to 15, wherein the composition is suitable for administration via a route selected from intravenous, intraarterial, subcutaneous, transdermal, intradermal, intramuscular, intraperitoneal, nasal, intracranial, intrathecal, intracardiac, intraosseous, transmucosal, oral, rectal, sublingual, topical, epidural, epicutaneous, inhalational, intraarticular, vaginal, auricular, and intravitreal.

Embodiment 17. The pharmaceutical composition of any one of embodiments 11 to 16, further comprising a second agent for the treatment of neuropathic pain.

Embodiment 18. The pharmaceutical composition of embodiment 17, wherein the second agent and/or the compound of Formula I is in a subtherapeutic amount.

Embodiment 19. The pharmaceutical composition of embodiment 17 or 18, wherein the second agent is an analgesic, anticonvulsant, tricyclic antidepressant, selective serotonin-norepinephrine reuptake inhibitor, topical anesthetic agent, nonsteroidal anti-inflammatory drug, antiarrhythmics, narcotic analgesic, or opioid.

The following examples are offered by way of illustration and not by way of limitation.

### Example 1 - Investigating the Effect of PAMs and Agonists on G Protein Activation by CB₁R

The effects of newly synthesized compounds on G protein activation were evaluated using the Glo assay as described in FIG. 2. These racemate PAMs of CB₁R were screened at a fixed micromolar concentration for their ability to activate/couple to G-protein (Gi) independently or in combination with an orthosteric ligand (a CB1R agonist CP 55,940). Activation of G-protein by the ligand-bound receptor can be estimated by reduction in the Luciferase intensity.

### Example 2 - Luciferase Assay for Evaluating GTP-Turnover

As shown in FIG. 2, in this assay, first (1) receptor and/or G-protein are added to the solution with GTP, and G-protein hydrolyzes GTP. Second (2), enzyme (nucleoside-diphosphate kinase) and ADP are added, and the enzyme converts GTP and ADP to GDP and ATP. Next, (3) Luciferin is added, and luciferase emits light using ATP in the solution. Weaker luminescence signal indicates better GTPase activity which in turn means better receptor activation. This assay can be used to evaluate the ligand efficacy at the receptor. For example, full agonist will give a much lower luminescence compared to partial agonist and adding PAMs will further decrease the luminescence of agonists.

### Example 3 - Identification of PAMs Suitable for Treating Neuropathic Pain

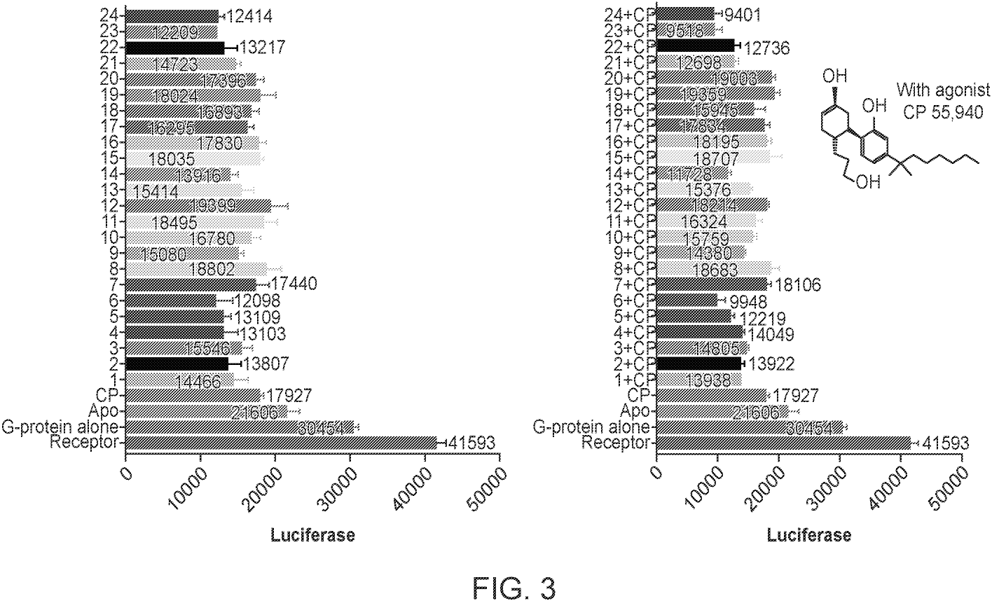

As shown in FIG. 3, most of the newly synthesized PAMs increased GTP-turnover independently of the agonist (orthosteric ligand) (FIG. 3, lower the bar higher the GTP-turnover). This suggests that the PAMs are able to bind the orthosteric binding site in addition to the allosteric site. Compounds that enhance CP 55,940-mediated G protein activation were further screened at additional concentrations to determine the optimum activation concentration, and those producing no GTP turn-over identified as PAMs.

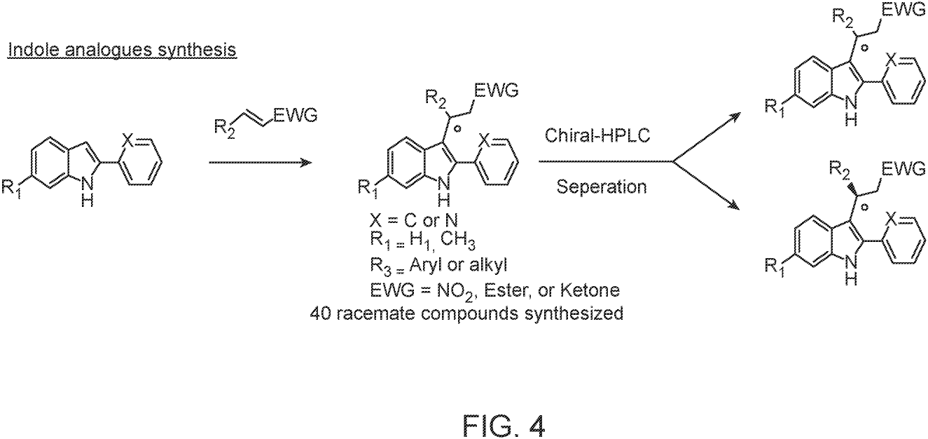

Further, the possibility of an enantiospecific allosteric modulation of CB₁R was tested, i.e. testing for one enantiomer engaged the allosteric site specifically and the other bound both the allosteric and orthosteric sites. To test this variable binding of the enantiomers, specific enantiomers were prepared via chiral-HPLC separation (FIG. 4).

### Example 4 - Identifying Agonists and PAMs Bias for β-Arrestin Recruitment by CB₁R

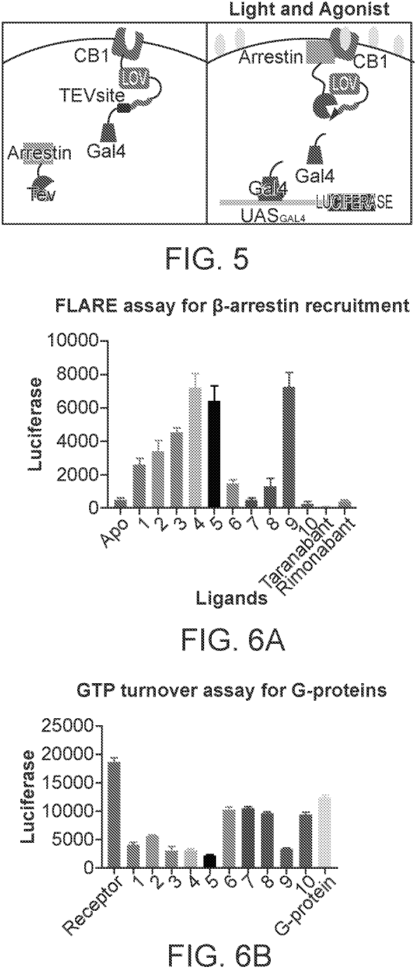

To explore the possibility of the CB₁R ligands that are biased towards arrestin signaling, the PathHunter GPCR β-arrestin assay (described below, FIG. 5) was used to study β-arrestin recruitment. In this assay, cells expressing CB₁R with a small enzyme donor fragment are co-expressed with a fusion protein of β-arrestin and the larger N-terminal deletion mutant of β-galactosidase.

### Example 5 - β-Arrestin Recruitment Assay

HEK293T cells in 6-well plates were transfected with 150 ng UAS-luciferase, 350 ng FLARE transcription factor (TF) component, 100 ng FLARE protease component with polyethylenimine (PEI) for about 8 hours and then each well is diluted in 6 ml media and 200 µL added to each well (96 wells). Cells were kept in dark by wrapping the plates in aluminium foil. Plates were incubated at 37° C. under 5% CO₂. Subsequent procedures were performed in a dark room with red light illumination. Cells were allowed to settle for an additional 9-12 hours before stimulation. For drug stimulation, 50 µL of media were added with the ligand (5x) to the final concentration indicated. For conditions without drug stimulation, 50 µL of complete growth media was added. After Dark plate, the cells were rewrapped in aluminium foil and for light illuminate light. After light stimulation, incubated at 37° C. under 5% CO₂ for 6-9 hours until the luciferase assay.

Media was then aspirated from each well in the 96-well plate and 100 µL DPBS was added to each well to wash. After aspirating PBS, 50 µL of the Glo lysis reagent was added. The plate was shaken till cells were detached from surface. In a 384 well plate, 10 µL of lysed cells and 10 µL of luciferase reagent was added and analysed using a plate reader.

Activation of CB₁R by agonists stimulates β-arrestin recruitment by CB₁R, resulting in the formation of an active β-galactosidase enzyme; an interaction that can be detected using a chemiluminescent reagent. Agonist-PAM combinations assessed in the Glo assay was evaluated in the β-arrestin assay, allowing a direct comparison of PAM signaling bias. As data shows ten ligands from Glo assay were examined for biased enhancement of G-protein coupling with no coupling of arrestin 2 via a transcription readout (Luciferase) assay. Thus, compounds are identified that acted as a full agonist for Gi coupling but only as a partial agonist for β-arrestin 2 coupling.

### REFERENCES

1. Treede, R. D.; Jensen, T. S.; Campbell, J. N.; et al., Neuropathic pain: redefinition and a grading system for clinical and research purposes. Neurology 2008, 70 (18), 1630-5.
2. Yawn, B. P.; Wollan, P. C.; Weingarten, T. N.; et al., The prevalence of neuropathic pain: clinical evaluation compared with screening tools in a community population. Pain Med 2009, 10 (3), 586-93.
3. Berger, A.; Dukes, E. M.; Oster, G., Clinical characteristics and economic costs of patients with painful neuropathic disorders. J Pain 2004, 5 (3), 143-9.
4. McCarberg, B. H.; Billington, R., Consequences of neuropathic pain: quality-of-life issues and associated costs. Am J Manag Care 2006, 12 (9 Suppl), S263-8.
5. Gaskin, D. J.; Richard, P., The economic costs of pain in the United States. J Pain 2012, 13 (8), 715-24.
6. McQuay, H. J.; Tramer, M.; Nye, B. A.; et al., A systematic review of antidepressants in neuropathic pain. Pain 1996, 68 (2-3), 217-27.
7. Bosier, B.; Muccioli, G. G.; Hermans, E.; Lambert, D. M., Functionally selective cannabinoid receptor signalling: therapeutic implications and opportunities. Biochem Pharmacol 2010, 80 (1), 1-12.
8. Raehal, K. M.; Bohn, L. M., Beta-arrestins: regulatory role and therapeutic potential in opioid and cannabinoid receptor-mediated analgesia. Handb Exp Pharmacol. 2014, 219, 427-43.
9. Pertwee, R. G., Cannabinoid receptors and pain. Prog Neurobiol 2001, 63 (5), 569-611.
10. Rahn, E. J.; Hohmann, A. G., Cannabinoids as pharmacotherapies for neuropathic pain: From the bench to the bedside. Neurotherapeutics 2009, 6 (4), 713-37.
11. Russo, E. B.; Hohmann, A. G., Role of cannabinoids in pain management. In Treatment of Chronic Pain by Medical Approaches, Deer, R. T.; Leong, S. M.; Gordin, V., Eds. Springer New York: New York, NY, 2015; pp 179-195. 12. Ashton, J. C.; Milligan, E. D., Cannabinoids for the treatment of neuropathic pain: clinical evidence. Curr Opin Investig Drugs 2008, 9 (1), 65-75.
13. Martin-Sanchez, E.; Furukawa, T. A.; Taylor, J.; et al., Systematic review and meta analysis of cannabis treatment for chronic pain. Pain Med2009, 10 (8), 1353-68.
14. Howlett, A. C., Cannabinoid receptor signaling. Handb Exp Pharmacol 2005, (168), 53-79.
15. Turu, G.; Hunyady, L., Signal transduction of the CB1 cannabinoid receptor. J Mol Endocrinol 2010, 44 (2), 75-85.
16. Ignatowska-Jankowska, B. M.; Baillie, G. L.; Kinsey, S.; et al., A cannabinoid CB receptor positive allosteric modulator reduces neuropathic pain in the mouse with no psychoactive effects. Neuropsychopharmacology 2015, 40 (13), 2948-59.
17. Price, M. R.; Baillie, G. L.; Thomas, A.; et al., Allosteric modulation of the cannabinoid CB1 receptor. MolPharmacol 2005, 68 (5), 1484-95.
18. Whalen, E. J.; Rajagopal, S.; Lefkowitz, R. J., Therapeutic potential of β-arrestin-and G protein-biased agonists. Trends MolMed2011, 17 (3), 126-39.
19. Joucla, L.; Djakovitch, L., Transition metal-catalysed, direct and site-selective N1-, C2-or C3-arylation of the indole nucleus: 20 Years of improvements. Adv Synth Catal 2009, 351 (5), 673-714.
20. Herrera, R. P.; Sgarzani, V.; Bernardi, L.; et al., Catalytic enantioselective Friedel-Crafts alkylation of indoles with nitroalkenes by using a simple thiourea organocatalyst. Angew. Chem. Int. Ed. Engl. 2005, 44 (40), 6576-9.
21. Lu, S.-F.; Du, D.-M.; Xu, J., Enantioselective Friedel-Crafts alkylation of indoles with nitroalkenes catalyzed by bifunctional tridentate bis(oxazoline)-Zn(II) complex. Org Lett 2006, 8 (10), 2115-8.
22. Ballini, R.; Petrini, M., Recent synthetic developments in the nitro to carbonyl conversion (Nef reaction). Tetrahedron 2004, 60 (5), 1017-47.

The preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1. A method for treating neuropathic pain in a subject, comprising administering to the subject a therapeutically effective amount of a compound of Formula I: 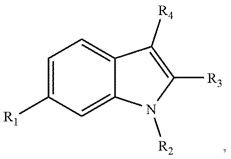
wherein:
R₁ is H, C₁-C₆ alkyl, aliphatic, alkoxy, amide, amine, thioether, haloalkyl, nitro, halogen, cycloaliphatic, or aryl;
R₂ is H, C₁-C₆ alkyl, aliphatic, alkoxy, amide, amine, thioether, haloalkyl, nitro, halogen, cycloaliphatic, or aryl;
R₃ is H, C₁-C₆ alkyl, or substituted or unsubstituted monocyclic or bicyclic aromatic ring containing 5 to 10 carbon atoms, wherein, one or more carbon atoms are replaced with one or more of N, O, or S; and
R₄ is C₁-C₆ nitro-alkyl, C₁-C₆ alkyl-amine, C₁-C₆ ketone, C₁-C₆ aldehyde, or C₁-C₆ carbonyl, wherein one or more carbons of the alkyl chain is substituted with substituted or unsubstituted monocyclic or bicyclic aromatic ring containing 5 to 10 carbon atoms, wherein, one or more carbon atoms are replaced with one or more of N, O, or S; and
wherein, the monocyclic or bicyclic aromatic ring is further optionally substituted with one or more groups selected from halogen, C₁-C₆ alkyl, alkoxy, carbonyl, amine, alkyl-amine, nitro, and nitro-alkyl.

2. The method of claim 1, wherein in the compound of Formula I:
R₁ is H or C₁-C₆ alkyl;
R₂ is H or C₁-C₆ alkyl;
R₃ is H, C₁-C₆ alkyl, pyridinyl, or phenyl; and
R₄ is nitro-alkyl, ketone, or aldehyde.

3. The method of claim 1 , wherein in the compound of Formula I the monocyclic or bicyclic aromatic ring containing 5 to 10 carbon atoms in the R₃ or R₄ position is phenyl, naphthyl, pyridine, quinoline, isoquinoline, pyrazine, quinoxaline, pyrimidine, quinazoline, pyridazine, cinnoline, phthalazine, triazine, furan, benzofuran, isobenzofuran, pyrrole, indole, isoindole, thiophene, benzothiophene, imidazole, benzimidazole, purine, pyrazole, indazole, oxazole, benzoxazole, isoxazole, benzisoxazole, thiazole, or benzothiazole.

4. The method of claim 1 , wherein in the compound of Formula I:
R₁ is H or methyl;
R₂ is H or methyl;
R₃ is H, methyl, pyridinyl, or phenyl; and
R₄ is:
nitro-ethyl substituted with phenyl, furan, or thiophene, optionally substituted with halogen or alkoxy; or
2-butanone, substituted with phenyl, furan, or thiophene, optionally substituted with halogen or alkoxy.

5. The method of claim 1 , wherein the compound is as shown in FIG. 1.

6. The method of claim 1 , wherein the neuropathic pain is caused by an autoimmune disease, diabetes, infection, inherited disorder, tumor, bone marrow disorder, kidney disease, liver disease, connective tissue disorder, underactive thyroid (hypothyroidism), trauma or pressure on the nerve, or vitamin deficiency.

7. The method of claim 1 , comprising administering the compound via a route selected from intravenous, intraarterial, subcutaneous, transdermal, intradermal, intramuscular, intraperitoneal, nasal, intracranial, intrathecal, intracardiac, intraosseous, transmucosal, oral, rectal, sublingual, topical, epidural, epicutaneous, inhalational, intraarticular, vaginal, auricular, and intravitreal.

8. The method of claim 1 , further comprising administering to the subject a second agent for the treatment of neuropathic pain.

9. The method of claim 8, comprising administering the second agent in a subtherapeutic amount and/or administering the compound of Formula I in a subtherapeutic amount.

10. The method of claim 8 , wherein the second agent is an analgesic, anticonvulsant, tricyclic antidepressant, selective serotonin-norepinephrine reuptake inhibitor, topical anesthetic agent, nonsteroidal anti-inflammatory drug, antiarrhythmics, narcotic analgesic, or opioid.

11. A pharmaceutical composition comprising a compound of Formula I and a pharmaceutically acceptable carrier, wherein the compound of Formula I comprises: 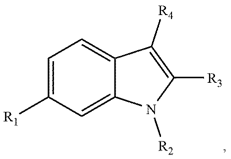
wherein:
R₁ is H, C₁-C₆ alkyl, aliphatic, alkoxy, amide, amine, thioether, haloalkyl, nitro, halogen, cycloaliphatic, or aryl;
R₂ is H, C₁-C₆ alkyl, aliphatic, alkoxy, amide, amine, thioether, haloalkyl, nitro, halogen, cycloaliphatic, or aryl;
R₃ is H, C₁-C₆ alkyl, or substituted or unsubstituted monocyclic or bicyclic aromatic ring containing 5 to 10 carbon atoms, wherein, one or more carbon atoms are replaced with one or more of N, O, or S; and
R₄ is C₁-C₆ nitro-alkyl, C₁-C₆ alkyl-amine, C₁-C₆ ketone, C₁-C₆ aldehyde, or C₁-C₆ carbonyl, wherein one or more carbons of the alkyl chain is substituted with monocyclic or bicyclic aromatic ring containing 5 to 10 carbon atoms, wherein, one or more carbon atoms are replaced with one or more of N, O, or S, and
wherein, the monocyclic or bicyclic aromatic ring is further optionally substituted with one or more groups selected from halogen, C₁-C₆ alkyl, alkoxy, carbonyl, amine, alkyl-amine, nitro, and nitro-alkyl.

12. The pharmaceutical composition of claim 11, wherein in the compound of Formula I:
R₁ is H or C₁-C₆ alkyl;
R₂ is H or C₁-C₆ alkyl;
R₃ is H, C₁-C₆ alkyl, pyridinyl, or phenyl; and
R₄ is nitro-alkyl, ketone, or aldehyde.

13. The pharmaceutical composition of claim 11 , wherein in the compound of Formula I the monocyclic or bicyclic aromatic ring containing 5 to 10 carbon atoms in the R₃ or R₄ position is phenyl, naphthyl, pyridine, quinoline, isoquinoline, pyrazine, quinoxaline, pyrimidine, quinazoline, pyridazine, cinnoline, phthalazine, triazine, furan, benzofuran, isobenzofuran, pyrrole, indole, isoindole, thiophene, benzothiophene, imidazole, benzimidazole, purine, pyrazole, indazole, oxazole, benzoxazole, isoxazole, benzisoxazole, thiazole, or benzothiazole.

14. The pharmaceutical composition of claim 11 , wherein in the compound of Formula I:
R₁ is H or methyl;
R₂ is H or methyl;
R₃ is H, methyl, pyridinyl, or phenyl; and
R₄ is:
nitro-ethyl substituted with phenyl, furan, or thiophene, optionally substituted with halogen or alkoxy; or
2-butanone, substituted with phenyl, furan, or thiophene, optionally substituted with halogen or alkoxy.

15. The pharmaceutical composition of claim 11 , wherein the compound is as shown in FIG. 1.

16. The pharmaceutical composition of claim 11 , wherein the composition is suitable for administration via a route selected from intravenous, intraarterial, subcutaneous, transdermal, intradermal, intramuscular, intraperitoneal, nasal, intracranial, intrathecal, intracardiac, intraosseous, transmucosal, oral, rectal, sublingual, topical, epidural, epicutaneous, inhalational, intraarticular, vaginal, auricular, and intravitreal.

17. The pharmaceutical composition of claim 11 , further comprising a second agent for the treatment of neuropathic pain.

18. The pharmaceutical composition of claim 17, wherein the second agent and/or the compound of Formula I is in a subtherapeutic amount.

19. The pharmaceutical composition of claim 17 wherein the second agent is an analgesic, anticonvulsant, tricyclic antidepressant, selective serotonin-norepinephrine reuptake inhibitor, topical anesthetic agent, nonsteroidal anti-inflammatory drug, antiarrhythmics, narcotic analgesic, or opioid.
